(19) United States
(12) Patent Application Publication
(10) Pub. No.: US 2020/0029869 A1
(43) Pub. Date: Jan. 30, 2020

(54) NONENZYMATIC DETERMINATION OF GLUCOSE AT NEAR NEUTRAL PH VALUES BASED ON THE USE OF NAFION AND PLATINUM BLACK COATED MICRONEEDLE ELECTRODE ARRAY

(71) Applicant: GACHON UNIVERSITY OF INDUSTRY-ACADEMIC COOPERATION FOUNDATION, Seongnam-si (KR)

(72) Inventors: Sungbo CHO, Seongnam-si (KR); Somasekhar R. CHINNADAYYALA, Incheon (KR)

(73) Assignee: GACHON UNIVERSITY OF INDUSTRY-ACADEMIC COOPERATION FOUNDATION, Seongnam-si (KR)

(21) Appl. No.: 16/520,776

(22) Filed: Jul. 24, 2019

(30) Foreign Application Priority Data
Jul. 24, 2018 (KR) 10-2018-0086167
Jul. 22, 2019 (KR) 10-2019-0088490

(51) Int. Cl. A61B 5/145; A61B 5/1486

(52) U.S. Cl. CPC A61B 5/14532; A61B 5/14865; A61B 5/14514

(57) ABSTRACT

Provided are a microneedle electrode sensor for nonenzymatic monitoring of blood sugar, a method of manufacturing the sensor, and a method of nonenzymatically measuring blood sugar using the sensor. The method of manufacturing the microneedle electrode sensor includes (a) forming multiple microneedle shapes in a substrate, (b) coating the entire area of the substrate with a passivation agent, and (c) coating a microneedle reactive portion disposed at a tip of each microneedle with a glucose oxidation catalyst.

### BACKGROUND OF THE INVENTION

### 1. Field of the Invention

The present invention relates to a microneedle electrode sensor for nonenzymatically measuring blood sugar and a method of manufacturing same. More particularly, the present invention relates to a microneedle electrode sensor capable of nonenzymatically measuring blood sugar by using platinum black as a metal catalyst rather than glucose oxidase and a method of manufacturing same.

### 2. Description of the Related Art

As of 2018, it is estimated that around 400 million people suffer from diabetes worldwide, and the number is estimated to reach 600 million by 2035. Blood sugar monitoring is essential for the treatment of diabetes mellitus and prevention of complications resulting from diabetes. To this end, the most common method of blood sugar monitoring is to measure blood sugar by sampling a small amount of blood from the fingertip of the patient. However, frequent blood sampling from the fingertip creates trypanophobia for many diabetes patients. Thus, there have been numerous investigations to find an alternative blood sugar monitoring method capable of avoiding frequent blood sampling from the fingertip. Microelectromechanical system (MEMS) technologies were ideally positioned to provide a variety of options for painless blood sampling. Microneedles are micro-sized devices, which have the ability to physically disrupt the outer layer of the skin with less pain and can be utilized for a variety of therapeutic and diagnostic systems. The interest in microneedles has grown rapidly and significantly owing to their potential for painless sampling and delivery to the intradermal space.

To date, microneedles have been used for the injection of insulin and as sensors for a variety of analytes. Most applications of microneedles have focused on minimally invasive pain-free transdermal delivery of drugs. Many sensing applications of microneedles have been reported, focusing primarily on the minimally invasive amperometric monitoring of glucose.

There have been attempts to use microneedles for amperometric glucose sensing. A case study was reported on wearable amperometric microneedle glucose sensor which was fabricated using MEMS techniques. Many glucose sensors have been reported that use enzymatic electrochemical sensing methods, such as smart patches that detect glucose electrically and devices that detect glucose using enzymatic electrochemical electrodes located beneath a microneedle array. However, these enzymatic glucose sensors have a problem of insufficient stability because they are susceptible to temperature, pH, humidity, etc.

On the other hand, unlike the enzymatic glucose sensors, nonenzymatic glucose sensors that do not use an enzyme have several advantages of high stability, simple manufacturing method, high reproducibility of blood sugar measurement results, and free from oxygen limitations. In addition, over the last few decades, the glucose measurement sensitivity of nonenzymatic glucose sensors has increased considerably to close to that of enzymatic sensors. Therefore, nonenzymatic glucose sensors have been suggested as an alternative to glucose level detection sensors.

### DOCUMENT OF RELATED ART

### Non-patent Document

(Non-patent Document 1) Mo R, Jiang T, Di J, Tai W, Gu Z, (2014) Emerging micro- and nano-technology based synthetic approaches for insulin delivery.
(Non-patent Document 2) Chem Soc Rev 43(10):3595-3629, Yoon Y, Lee G S, Yoo K, Lee J-B, (2013) Fabrication of a microneedle/CNT hierarchical micro/nano surface electrochemical sensors and its in-vitro glucose sensing characterization Sensors, 13(12):16672-16681

### SUMMARY OF THE INVENTION

An objective of the present invention is to provide a microneedle electrode sensor capable of nonenzymatically measuring blood sugar, a method of manufacturing the sensor, and a method of nonenzymatically measuring blood sugar using the sensor.

In order to achieve the above objective, the present invention provides a method of manufacturing a microneedle electrode sensor for nonenzymatically measuring blood sugar.

The microneedle electrode sensor for nonenzymatically measuring blood sugar includes: (a) forming multiple microneedle shapes from a substrate; (b) bending the microneedle shapes by 90° with respect to a principal surface of the substrate to form microneedles; (c) protecting reactive portions located at tips of the respective microneedles and a substrate contact pad of an electrode formed on the substrate with a shielding agent and coating the entire area of the substrate with a passivation agent; (d) removing the shielding agent from the reactive portions of the microneedles and from the substrate contact pad after performing the protecting of the reactive portions; and (e) coating the reactive portions of the microneedles with a glucose oxidation catalyst.

The bending of the microneedle shapes may include plating a surface of each of the microneedles with gold (Au), platinum (Pt), or silver (Ag).

The shielding agent for protecting the reactive portions of the respective microneedles may be polydimethylsiloxane (PDMS), the shielding agent for protecting the substrate contact pad may be parafilm.

The passivation agent may be parylene and may form a passivation layer having a thickness of 1 μm or more.

The glucose oxidation catalyst used in the coating of the reactive portions of the microneedles may be Pt black that is formed from an electrodeposition solution composed of 2.5% chloroplatinic acid, 0.05% lead acetate, and 0.1 M hydrochloric acid (HCl).

In addition, in the coating of the reactive portions of the microneedles, silver chloride may be used instead of the glucose oxidation catalyst to form a reference electrode.

The method may further include forming a Nafion layer on a surface of the glucose oxidation catalyst.

In order to achieve another objective, the present invention provides a microneedle electrode sensor for nonenzymatically sensing blood sugar.

The microneedle electrode sensor includes: a conductive metal substrate; multiple microneedles being erect with respect to the substrate; reactive portions disposed at tips of the respective microneedles; and multiple supports connecting the respective reactive portions to the substrate, in which the reactive portions are coated with a glucose oxidation catalyst and the supports and the substrate are coated with a passivation agent.

The microneedles may be coated with gold (Au), platinum (Pt), or silver (Ag).

The glucose oxidation catalyst may be Pt black.

The passivation agent may be parylene and may form a layer having a thickness of 1 μm or more.

Silver chloride may be used instead of the glucose oxidation catalyst to form a reference electrode.

Each of the reactive portions may further include a Nafion layer coated on the glucose oxidation catalyst.

In order to accomplish a further objective of the present invention, there is provided a nonenzymatic glucose sensing method of measuring an electrical signal representing a glucose level using a nonenzymatic microneedle electrode glucose sensor having a three-electrode configuration.

According to the present invention as described above, the microneedle electrode glucose sensor is prepared by coating the tip of each microneedle sequentially with Pt black and Nafion and coating the remaining portion of the microneedle with parylene. Thus, the microneedle electrode glucose sensor according to the present invention has improved stability and durability as compared with a conventional enzyme-based microneedle electrode glucose sensor. In addition, with the microneedle electrode glucose sensor according to the present invention, it is possible to nonenzymatically measure blood sugar, thereby enabling painless and continuous glucose monitoring.

### BRIEF DESCRIPTION OF THE DRAWINGS

FIG. 1 is a diagram illustrating a method of manufacturing a microneedle electrode sensor according to one embodiment of the present invention.

FIG. 2 is a scanning electron microscopy (SEM) image according to Measurement Example 1.

FIG. 3 is a diagram illustrating a cyclic voltammogram (CV) according to Measurement Example 2.

FIG. 4 is a diagram illustrating a linear sweep voltammetry (LSV) graph according to Measurement Example 3.

FIG. 5 is a current-time curve according to Measurement Example 4.

FIG. 6 is a graph illustrating reproducibility according to Measurement Example 5.

FIG. 7 is a graph illustrating stability according to Measurement Example 5.

FIG. 8 is a current-time curve according to Measurement Example 6.

### DESCRIPTION OF THE PREFERRED EMBODIMENTS

Hereinafter, embodiments of the present invention will be described in detail.

In order to achieve the above objective, the present invention provides a method of manufacturing a microneedle electrode sensor for nonenzymatically measuring blood sugar.

The microneedle electrode sensor for nonenzymatically measuring blood sugar includes: the steps of (a) forming multiple microneedle shapes from a substrate; (b) bending the microneedle shapes by 90° with respect to a principal surface of the substrate to form microneedles; (c) protecting reactive portions located at tips of the respective microneedles and a substrate contact pad of an electrode formed on the substrate with a shielding agent and coating the entire area of the substrate with a passivation agent; (d) removing the shielding agent from the reactive portions of the microneedles and from the substrate contact pad after performing the protecting of the reactive portions; and (e) coating the reactive portions of the microneedles with a glucose oxidation catalyst.

The step (b) may include plating a surface of each microneedle with gold (Au), platinum (Pt), or silver (Ag).

The shielding agent for protecting the reactive portions of the respective microneedles may be polydimethylsiloxane (PDMS), the shielding agent for protecting the contact pads of the substrate may be Parafilm.

The passivation agent may be parylene and may form a passivation layer having a thickness of 1 μm or more.

In the coating of the reactive portions, silver chloride may be coated on the reactive portions instead of the glucose oxidation catalyst to form an Ag/AgCl reference electrode when the microneedle electrode sensor manufactured through the method described above has a three-electrode configuration.

The glucose oxidation catalyst used in the step (e) may be Pt black that is formed from an electrodeposition solution composed of 2.5% chloroplatinic acid, 0.05% lead acetate, and 0.1 M hydrochloric acid (HCl).

By performing the steps described above, it is possible to manufacture a Pt black microneedle electrode sensor.

The method may further include a step of forming a Nafion coating layer on the surface of the glucose oxidation catalyst after the step (e) is performed.

By performing the steps described above, it is possible to manufacture a Pt black/Nafion microneedle electrode sensor.

Specifically, in the microneedle electrode sensor manufactured according to the present invention, the microneedles formed in the step (a) serve as working electrodes. The substrate provided with the multiple microneedles is a conductive metallic substrate. More specifically, the substrate is a stainless steel substrate. The substrate in which the multiple microneedles are formed may be coated with a highly conductive metal such as gold (Au), platinum (Pt), or silver (Ag). The microneedles are erect with an angle of 90° with respect to the surface of the substrate. That is, the microneedles are perpendicular to the surface of the substrate. The microneedles are arranged in a 3×5 array.

In the step (c), the reactive portions of the microneedles are coated with polydimethylsoloxane (PDMS) gel for protection. The contact pad protruding from one side of the substrate provided with the microneedles is coated with parafilm for protection. The rest of the substrate may be coated with parylene which is a passivation agent.

The parylene coating as a passivation member exhibits insulating and water-repelling effects, thereby preventing the microneedle electrode sensor from being corroded by biological substances or preventing biofouling on the microneedle electrode sensor during the use of the microneedle electrode sensor. The parylene coating is formed through chemical vapor deposition. It is difficult to etch parylene, thus, according to the present invention, by forming PDMS and parafilm at the regions that are designed not to be covered with the parylene coating, it is possible to form the parylene coating without requiring post-etching after formation thereof. In addition, when the parylene coating has a thickness of 1 μm or more, it is kept stable when it is immersed in a liquid such as water for several days. However, the parylene coating with a thickness of less than 1 μm becomes unstable as soon as it is dipped in a liquid. Therefore, the parylene coating preferably has a thickness of 1 μm or more. Most preferably, the parylene coating has a thickness of 5 μm or more. However, when the parylene coating is excessively thick, it acts as a physical obstacle when it is inserted into the body through the skin layer. Therefore, it is preferable that the parylene coating has a thickness of 10 μm or less.

In the step (e), the glucose oxidation catalyst coated on the reactive portions of the microneedles may be Pt black. The Pt black coating used as catalyst for oxidation and reduction of glucose is formed after the PDMS on the reactive portions of the microneedle is removed. Specifically, in order to use a three-electrode system composed of an Ag/AgCl reference electrode, a Pt counter electrode, and a microneedle working electrode, the Pt black coating is formed at a current density of −2.5 mA/cm² from a 3 mL solution comprised of 2.5% chloroplatinic acid, 0.05% lead acetate, and 0.1 M hydrochloric acid (HCl). Pt black is in the form of platinum chloride salt that is easy to use for electroplating, and it is a representative electrode material which is abundantly present, among available electrode materials. Pt black has high biocompatibility and exhibits high catalytic activity to glucose oxidation.

Therefore, the use of Pt black enables glucose oxidation to occur in the absence of enzymes such as glucose oxidase while minimizing an approach of electrically active co-existing blockers thereto. In addition, since Pt black is an abundantly occurring material, the use of Pt black reduces the production cost of a microneedle electrode sensor.

After the step (e) is performed, a further step of forming a Nafion coating layer on the surface of the glucose oxidation catalyst-coated reactive portion of the microneedle is performed. Nafion is a biocompatible material acting as a barrier that prevents several in vivo signal-interfering substances that are not involved in glucose and blood sugar from accessing the electrode surface (Pt black) using electrostatic repulsion. In this case, since a glucose oxidation catalyst such as Pt black is coated on the reactive portion of the microneedle, the Nafion coating layer may be formed by a simple method of dipping the reactive portion of the microneedle in a Nafion solution. Specifically, in order to form the Nafion coating layer, the reactive portion of the microneedle is immersed for 60 seconds in a solution in which Nafion and ethanol are mixed in a volume ratio of 1:6, is then dried for 40 seconds on a hot plate heated to a temperature of 50° C., and is then dried with fresh air for 1 hour at room temperature (i.e., 24° C.)

In order to achieve another objective, the present invention provides a microneedle electrode sensor for nonenzymatically sensing blood sugar.

The microneedle electrode sensor for nonenzymatically sensing blood sugar includes: a conductive metal substrate; multiple microneedles being erect with respect to the surface of the substrate; reactive portions disposed at the tips of the respective microneedles; and multiple supports connecting the respective reactive portions to the substrate, in which the reactive portions are covered with a glucose oxidation catalyst layer and the supports and the substrate are covered with a passivation layer.

The microneedles are coated with gold (Au), platinum (Pt), or silver (Ag).

The passivation layer may be made from a passivation agent (for example, parylene) and may have a thickness of 1 μm or more.

The reactive portion is coated with silver chloride (AgCl) instead of the glucose oxidation catalyst layer to form a reference electrode.

The configuration described above may be the configuration of an Ag/AgCl microneedle reference electrode.

The glucose oxidation catalyst layer may be made from Pt black. The configuration described above may be the configuration of a Pt microneedle electrode sensor.

Each of the reactive portions may further include a Nafion coating layer formed on the glucose oxidation catalyst layer.

The configuration described above may be the configuration of a Pt black/Nafion microneedle electrode sensor.

In order to accomplish a further objective of the present invention, there is provided a method of nonenzymatically sensing an electrical signal representing a glucose level using a nonenzymatic microneedle electrode glucose sensor having a three-electrode configuration.

The three-electrode configuration includes a working electrode, a counter electrode, and a reference electrode, in which the working electrode is a Pt black/Nafion microneedle electrode sensor, the counter electrode is a Pt microneedle electrode sensor, and the reference electrode is an Ag/AgCl microneedle electrode sensor.

Hereinafter, the present invention will be described in more detail with reference to examples and measurement examples. These examples and measurement examples are only for illustrative purposes, and it will be apparent to those skilled in the art that the scope of the present invention is not limited thereto.

### Example 1

A Pt black/Nafion microneedle electrode sensor was prepared in a manner described below. First, a stainless steel substrate was patterned with a jet of wet ferric chloride (FeCl₃) chemical etchant under a pressure of 2 kgf/cm² for 60 seconds to form microneedles. Next, the surfaces of the microneedles were electroplated with a thin layer of gold (Au) and then bent by an angle of 90° with a jig. Next, the tip of each microneedle was shielded with PDMS which is a gel-type shielding agent, and the contact pad of the substrate provided with the microneedle electrodes was shielded with parafilm. Next, the other regions (i.e., except for the shielded regions) were covered with a 5 μm-thick parylene coating layer. Next, the PDMS and the parafilm were removed so that the tip of each microneedle and the contact pad were exposed. Next, Pt black was electrodeposited on the surface of the tip of each microneedle. For the Pt black electrodeposition, a three-electrode system (composed of an Ag/AgCl reference electrode, a Pt counter electrode, and a microneedle working electrode) was used. The Pt black was formed through electrodeposition from a 3 mL solution consisting of 2.5% chloroplatinic acid, 0.05% lead acetate, and 0.1 M hydrochloric acid (HCl). The electrodeposition was performed for 200 seconds at a current density of −2.5 mA/cm². Next, the Pt black-electrodeposited tip of each microneedle was dipped for 60 seconds in a mixture solution of Nafion and ethanol in a volume ratio of 1:6. Next, the microneedles were dried for 40 seconds on a hot plate heated to a temperature of 50° C. and then dried with fresh air for 1 hour at the room temperature (24° C.). Thus, a Pt black/Nafion microneedle electrode sensor was prepared.

### Example 2

A Pt microneedle electrode sensor was prepared in a manner described below. First, a stainless steel substrate was patterned with a jet of wet ferric chloride (FeCl₃) chemical etchant under a pressure of 2 kgf/cm² for 60 seconds to form microneedles. Next, the surfaces of the microneedles were electroplated with a thin layer of platinum (Pt), and the microneedles were bent by an angle of 90° with a jig. Next, the tip of each microneedle was shielded with PDMS which is a gel-type shielding agent, and the contact pad of the substrate provided with the microneedle electrodes was shielded with parafilm. The other regions except for the shielded regions were covered with a 5 μm-thick parylene coating layer.

### Example 3

An Ag/AgCl microneedle electrode sensor was prepared in a manner described below. First, a stainless steel substrate was patterned with a jet of wet ferric chloride (FeCl₃) chemical etchant under a pressure of 2 kgf/cm² for 60 seconds to form microneedles. Next, the surfaces of the microneedles were electroplated with a thin layer of silver (Ag), and the microneedles were bent by an angle of 90° with a jig. Next, the tip of each microneedle was shielded with PDMS which is a gel-type shielding agent, and the contact pad of the substrate provided with the microneedle electrodes was shielded with parafilm. Next, the other regions (i.e., except for the shielded regions) of the substrate were covered with a 5 μm-thick parylene coating layer. Next, the PDMS and the parafilm were removed from the surface of the tip of each microneedle and the surface of the contact pad of the substrate. Next, the surface of the tip of each microneedle was coated with silver chloride (AgCl) to form an Ag/AgCl microneedle electrode sensor.

### Measurement Example 1

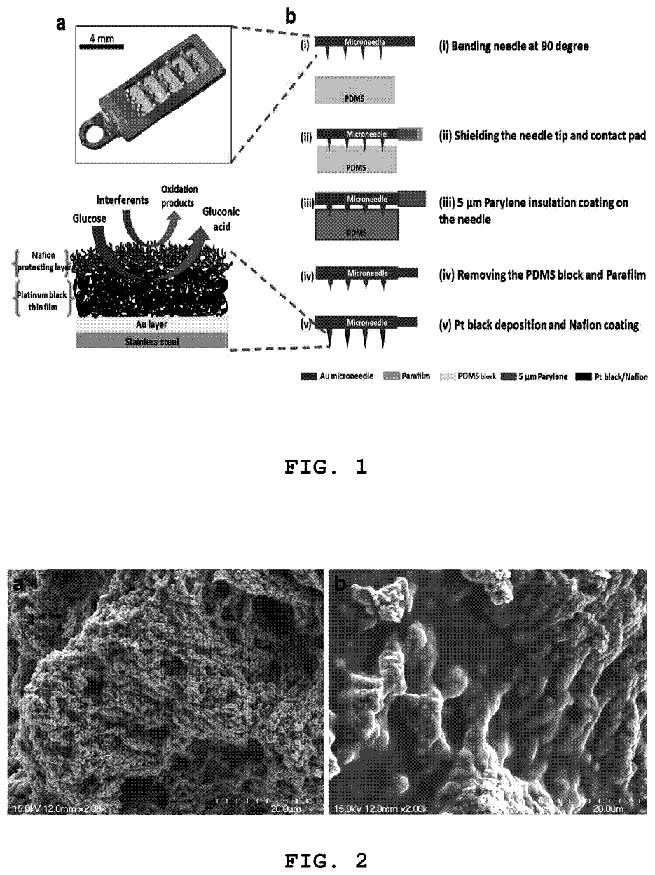

Scanning electron microscopy (SEM) images of microneedle electrode sensors were obtained. Referring to the SEM images, the surface of a microneedle not coated with platinum (pt) black is smooth. However, after the surface of a microneedle was coated with Pt black, the surface became rough and porous (see a in FIG. 2). As in the microneedle that was manufactured according to Example 1 and in which Nafion was coated on the surface of the platinum black coating, the thin film-coated reactive portion of the microneedle was observed (b in FIG. 2).

### Measurement Example 2

The Pt black/Nafion microneedle electrode prepared in Example 1 was used as a working electrode, the Pt microneedle electrode prepared in Example 2 was used as a counter electrode, and the Ag/AgCl microneedle electrode prepared in Example 3 was used as a reference electrode to measure an electric current at a predetermined voltage. Specifically, a three-electrode system (a in FIG. 3) composed of a working electrode, a counter electrode, and a reference electrode and a two-electrode system (b in FIG. 3) composed of a working electrode and a reference electrode were used for measurement. By measuring the current was measured in a buffered saline solution (pH 7.4) in a voltage range of 0.7 V to 0.4 and at a scanning speed of 50 mV/s, cyclic voltammograms (CVs) were obtained. In a and b in FIG. 3, the Pt black-coated microneedle electrode sensors exhibited typical Pt cyclic voltammograms and exhibited oxidation and reduction peaks higher than those of a conventional microneedle electrode sensor not coated with Pt black. This is because the impedance of the electrode decreases as Pt black increases a reactive area of the electrode. In the case of Nafion-coated (Pt black/Nafion) microneedle electrodes, the anodic peak current slightly increased at 0.12 V, for example, by 12% and 40% (a and b in FIG. 3, respectively) but significantly decreased at −0.37 V, for example, by 143% and 49% (a and b in FIG. 3, respectively). However, no change in the anodic peak voltage was found. This means that even after the Nafion film was formed, the characteristics of the microneedle electrode sensor measuring the redox current were not lost.

### Measurement Example 3

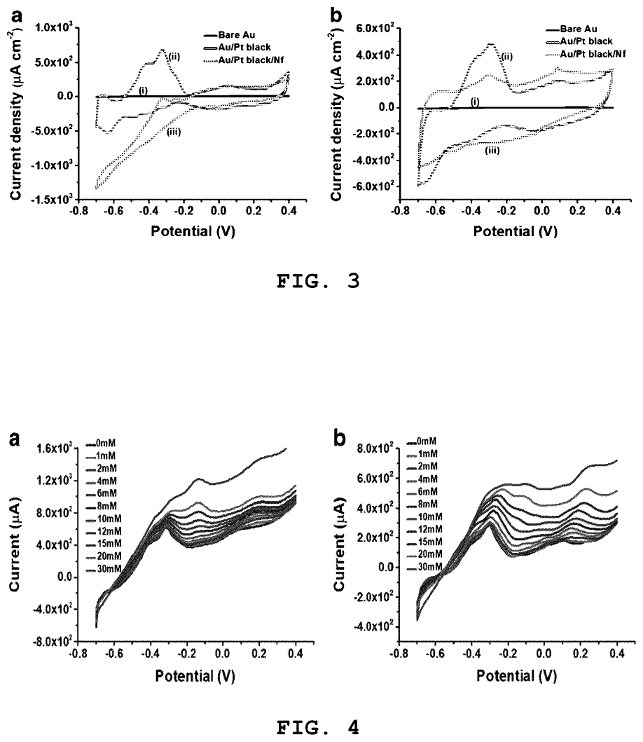

The Pt black/Nafion microneedle electrode prepared in Example 1 was used as a working electrode, the Pt microneedle electrode prepared in Example 2 was used as a counter electrode, and the Ag/AgCl microneedle electrode prepared in Example 3 was used as a reference electrode. With the use of a three-electrode system (a in FIG. 4) and a two-electrode system (b in FIG. 4), voltage and current were measured in buffered saline solutions (pH 7.4) having different concentrations of glucose ranging from 0 to 30 mM at a scanning speed of 50 mV/s. The measurements were plotted in a linear sweep voltammetry (LSV) graph shown in FIG. 4. The current peak at −0.37 V is attributed to the adsorbed intermediates formed for the electrosorption of glucose (adsorption-desorption zone of hydrogen: −1.0 V to −0.6 V). The second peak at −0.12 V and the third peak at +0.12 V are attributed to the direct oxidation of glucose on an electrode surface (glucose oxidation region: −0.4 V to +0.6 V).

As the glucose concentration increased, the current peaks due to glucose oxidation increased at the above-mentioned voltages. The optimum voltage for glucose oxidation was +0.12 V, and this low overvoltage allowed unobstructed measurements.

As to the glucose measurement voltage, conventional nonenzymatic electrochemical glucose sensors require a high measurement voltage (about +0.6 V), whereas the sensor of the present invention requires a low measurement voltage of +0.12 V. This reduction in voltage makes it possible to safely perform in vivo analysis and has the effect of reducing the power consumption of a portable sensor.

### Measurement Example 4

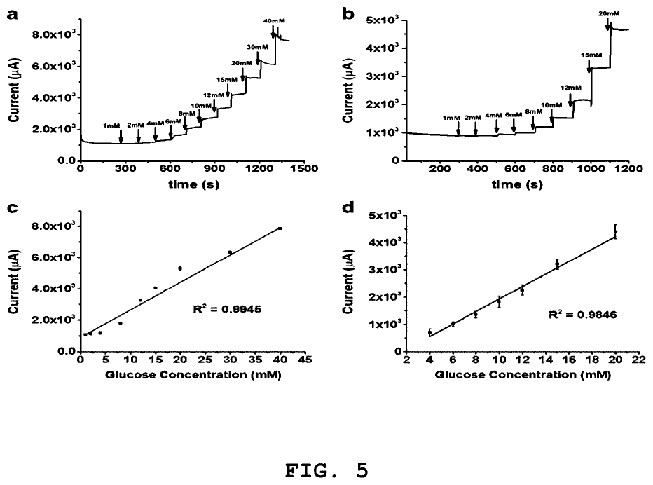

Glucose was continuously added at time intervals of 100 seconds while a voltage of +0.12 V is maintained. In this case, the Pt black/Nafion microneedle electrode prepared in Example 1 was used as a working electrode, the Pt microneedle electrode prepared in Example was used as a counter electrode, and the Ag/AgCl microneedle electrode prepared in Example 3 was used as a reference electrode to measure an electric current over time. In the case of a three-electrode system (a in FIG. 5), the anodic current increased linearly with an increasing glucose concentration and reached saturation at 40 mM. In the case of a two-electrode system (b in FIG. 5) the saturation was reached at 20 mM.

The three-electrode system exhibited a measurement sensitivity of 175±0.84 μA/mM·cm² in a broad dynamic range of 1 to 40 mM and the two-electrode system exhibited a measurement sensitivity of 205.57±0.84 μA/mM·cm² in a broad dynamic range of 4 to 20 mM. The response time of the sensors was 2 seconds and the limits of detection (DL) of glucose were 23±2.0 μm and 6.0±1.0 μm, respectively in the three-electrode system and the two-electrode system. The DL values are regarded the same as measurement sensitivities. The measurement results proved that the upper limits of measurement concentration and the measurement sensitivities of the glucose sensors according to the present invention were higher than those of existing nonenzymatic electrochemical glucose sensors.

The high linearity results over a wide concentration range demonstrated highly reproducible electrochemical operations of the manufactured sensors for glucose to be measured (c and d in FIG. 5). In this case, the measured glucose concentration range includes glucose concentrations determined by physiology and pathology. This range indicates that the microneedle sensor of the present disclosure is used as a glucose sensor for diagnostic purpose in the future. In terms of the relative performance of the sensors, the three-electrode system (c in FIG. 5) had better linear range, more appropriate sensitivity, and more appropriate DL than the two-electrode system (d in FIG. 5).

### Measurement Example 5

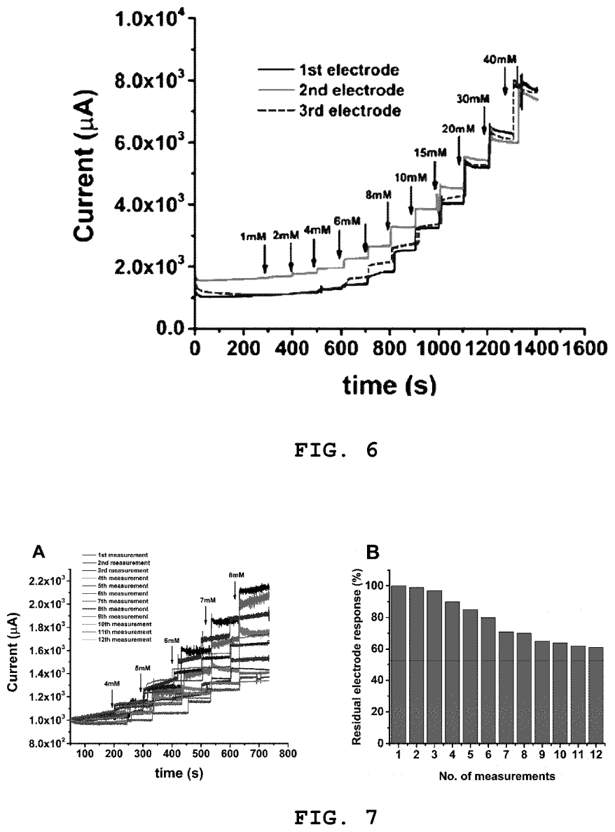

Reproducibility of each of the microneedle electrodes prepared in Examples 1 to 3 was evaluated by examining reactions at various glucose concentrations. A three-electrode system in which the Pt black/Nafion microneedle electrode prepared in Example 1 was used as a working electrode, the Pt microneedle electrode prepared in Example 2 was used as a counter electrode, and the Ag/AgCl prepared in Example 3 was used as a reference electrode exhibited a relative deviation of 3.2% which means an acceptable reproducibility when glucose was added to a 0.1 M PBS buffer (pH 7.4) continuously (see FIG. 6).

Stability was examined by continuously adding and measuring glucose in a concentration range of 4 to 8 mM to a 0.1 M PBS buffer (pH 7.4) under a constant voltage of +0.12 V for 5 hours. The result proved that 60% of the initial activity of the sensor was still maintained at the end of 12 consecutive measurements (A and B in FIG. 7).

On the other hand, existing nonenzymatic electrochemical glucose sensors can measure glucose in a basic environment such as NaOH solution. For this reason, it is not easy to use the existing nonenzymatic sensors for actual sample analysis. However, the Pt black/Nafion-based sensor according to the present invention can measure glucose even in a neutral environment. Therefore, it is easy to analyze actual samples with the sensor according to the present invention.

### Measurement Example 6

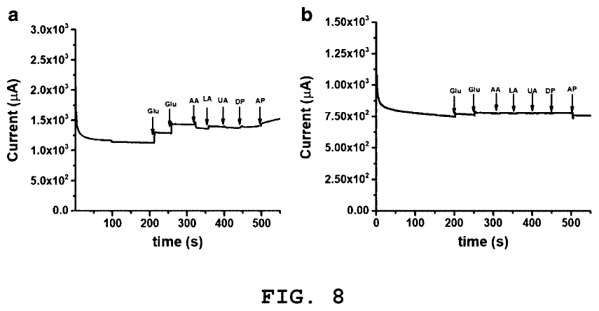

In order to investigate the selectivity to glucose for the microneedle electrode sensors manufactured by the methods of Examples 1 to 3, the effects of interference materials such as AA, DP, AP, LA and UA in actual blood samples were studied (AA: ascorbic acid, LA: lactic acid, UA: uric acid, DP: dopamine, and AP: acetaminophen). The concentration of interference materials in a sample used for investigation was 10 times higher than a normal physiological level. FIG. 8 shows experimental results of glucose measurements of Pt black/Nafion microneedle electrode sensors having a three-electrode system (a in FIG. 8) and a two-electrode system (b in FIG. 8), respectively, in samples in which interference materials are present in a concentration of 0.1 mM while glucose is present in a concentration of 2 mM. The results proved that the addition of any interference material had a negligible effect on the detection of glucose. This negligible influence of the interference materials is due to electrostatic repulsion caused by Nafion, which prevents the interference materials from approaching the surface of the microneedle electrode. The interference-free glucose detection is due to a strong and selective electrocatalytic activity of Pt with respect to glucose.

### Measurement Example 7

Glucose concentrations in three different blood samples (A, B, and C) were measured at 0.12 V through a standard addition method. For the measurements, the three-electrode microneedles prepared by the manufacturing methods of Examples 1 to 3 were used. As a comparative example, a commercially available glucometer was used. The results are shown in Table 1.

TABLE 1

| Microneedle electrode | |
|---|---|
| sensors of Examples 1 to 3 | Commercial glucometer |
| 3.8 ± 0.14 mM | 4.0 ± 0.016 mM |
| 5.1 ± 0.21 mM | 5.2 ± 0.012 mM |
| 4.7 ± 0.17 mM | 4.5 ± 0.031 mM |

This shows that there is no big difference between the two measured values. Compatibility between these volumetric measurements demonstrates reliability of the sensor of the present invention for measurement of glucose in real blood samples. This study indicates that the sensor of the present invention can be used for sensitive and quantitative glucose analysis on real samples.

Although specific parts of the present invention have been described in detail, it should be apparent to those skilled in the art that such specific descriptions merely present preferred embodiments and thus the scope of the present invention is not limited thereto. Thus, the substantial scope of the present invention will be defined by the appended claims and their equivalents.

1. A method of manufacturing a microneedle electrode sensor for nonenzymatically measuring blood sugar, the method comprising:
(a) forming multiple microneedle shapes in a substrate;
(b) coating the entire area of the substrate with a passivation agent; and
(c) coating a microneedle reactive portion disposed at a tip of each microneedle with a glucose oxidation catalyst.

2. The method according to claim 1, wherein the forming of the multiple microneedle shapes comprises plating the substrate with a conductive metal, and
the conductive metal is gold (Au), platinum (Pt), or silver (Ag).

3. The method according to claim 2, further comprising forming microneedles by bending the microneedle shapes such that the microneedles are erect with respect to the plated substrate.

4. The method according to claim 3, further comprising protecting, with a shielding agent, the microneedle reactive portion disposed at the tip of each microneedle and a substrate contact pad on which an electrode is to be formed.

5. The method according to claim 4, wherein the shielding agent for protecting the microneedle reactive portion of each respective microneedle is polydimethylsiloxane (PDMS), and the shielding agent for protecting the substrate contact pad is parafilm.

6. The method according to claim 5, wherein in the coating of the entire area of the substrate, the passivation agent is parylene.

7. The method according to claim 6, wherein in the coating the entire area of the substrate, the passivation agent is formed to be 1 μm or thicker.

8. The method according to claim 7, wherein after the coating the entire area of the substrate, removing the shielding agent covering the microneedle reactive portions and the shielding agent covering the substrate contact pad.

9. The method according to claim 8, wherein in the coating of the microneedle reactive portion, the glucose oxidation catalyst is platinum black.

10. The method according to claim 7, wherein in the coating of the microneedle reactive portion, the microneedle reactive portion is coated with silver chloride (AgCl) instead of the glucose oxidation catalyst to form a reference electrode.

11. The method according to claim 9, further comprising coating a surface of the glucose oxidation catalyst with Nafion after the coating of the microneedle reactive portion with the glucose oxidation catalyst.

12. A microneedle electrode sensor for nonenzymatic monitoring of blood sugar, the sensor comprising:
a conductive metal substrate;
multiple microneedles being erect with respect to the substrate;
reactive portions at tips of the respective microneedles; and
supports that connect the respective reactive portions to the substrate,
wherein the reactive portions are coated with a glucose oxidation catalyst, and
the supports and the substrate are coated with a passivation agent.

13. The sensor according to claim 12, wherein a surface of the substrate and the surfaces of the microneedles are plated with a conductive metal.

14. The sensor according to claim 13, wherein the conductive metal is gold (Au), platinum (Pt), or silver (Ag).

15. The sensor according to claim 14, wherein the passivation agent is parylene.

16. The sensor according to claim 15, wherein the passivation agent is formed to be 1 μm or thicker.

17. The sensor according to claim 16, wherein the glucose oxidation catalyst is platinum black.

18. The sensor according to claim 17, wherein the reactive portion may further include a Nafion coating layer formed on a coating layer made of the glucose oxidation catalyst.

19. The sensor according to claim 16, wherein the reactive portion is coated with silver chloride (AgCl) instead of the glucose oxidation catalyst to form a reference electrode.

20. A nonenzymatic blood sugar sensing method of measuring an electrical signal representing a glucose level using the two-electrode or three-electrode microneedle electrode sensor according to claim 17.
